(19) United States
(12) Patent Application Publication
(10) Pub. No.: US 2014/0288369 A1
(43) Pub. Date: Sep. 25, 2014

(54) MECHANICAL IMAGE ROTATION FOR RIGIDLY COUPLED IMAGE SENSOR AND ENDOSCOPE

(71) Applicant: Olive Medical Corporation, Salt Lake City, UT (US)

(72) Inventors: Jeremiah D. Henley, Midvale, UT (US); Brian Dean, Salt Lake City, UT (US)

(73) Assignee: Olive Medical Corporation, Salt Lake City, UT (US)

(21) Appl. No.: 14/214,328

(22) Filed: Mar. 14, 2014

Related U.S. Application Data
(60) Provisional application No. 61/791,629, filed on Mar. 15, 2013.

(51) Int. Cl. A61B 1/00; A61B 1/07; A61B 1/05

(52) U.S. Cl. CPC A61B 1/00096; A61B 1/05; A61B 1/07
USPC 600/109

(57) ABSTRACT

The disclosure extends to endoscopic devices and systems for image rotation for a rigidly coupled image sensor. The disclosure allows for a distal prism to rotate, which changes the angle of view of the user or operator, while the sensor remains fixed at a constant location. This allows the device to be used in the same manner as expected by a user or operator. The user or operator may rotate an outer lumen, thereby changing the angle of view, while the sensor remains in a fixed position and the image viewable on screen remains at a constant horizon. The prism may rotate while the sensor does not rotate, such that the user does not lose orientation.

### CROSS-REFERENCE TO RELATED APPLICATIONS

This application claims the benefit of U.S. Provisional Application No. 61/791,629, filed Mar. 15, 2013, which is hereby incorporated by reference herein in its entirety, including but not limited to those portions that specifically appear hereinafter, the incorporation by reference being made with the following exception: In the event that any portion of the above-referenced provisional application is inconsistent with this application, this application supersedes said above-referenced provisional application.

### STATEMENT REGARDING FEDERALLY SPONSORED RESEARCH OR DEVELOPMENT

Not Applicable.

### BACKGROUND

Advances in technology have provided advances in imaging capabilities for medical use. One area that has enjoyed some of the most beneficial advances is that of endoscopic surgical procedures because of the advances in the components that make up an endoscope.

Conventional endoscopes used in, e.g., arthroscopy and laparoscopy are designed such that the image sensors are placed at the proximal end of the device, within the hand-piece unit. In such a configuration, the endoscope unit must transmit the incident light along its length toward the sensor via a complex set of precisely coupled optical components, with minimal loss and distortion. The cost of the endoscope unit is dominated by the optics, since the components are expensive and the manufacturing process is labor intensive. Moreover, this type of scope is mechanically delicate and relatively minor impacts can easily damage the components or upset the relative alignments thereof. This necessitates frequent, expensive repair cycles in order to maintain image quality.

One solution to this issue is to place the image sensor within the endoscope itself at the distal end, thereby potentially approaching the optical simplicity, robustness and economy that are universally realized within, e.g., cell phone cameras. An acceptable solution to this approach is by no means trivial, however, as it introduces its own set of engineering challenges, not the least of which is the fact that the sensor must fit within a highly confined area.

Placing aggressive constraints on sensor area naturally pushes one in the direction of fewer and/or smaller pixels. Lowering the pixel count directly affects the spatial resolution. Reducing the pixel area reduces the available signal capacity and the sensitivity. Lowering the signal capacity reduces the dynamic range i.e. the ability of the camera to simultaneously capture all of the useful information from scenes with large ranges of luminosity. There are various methods to extend the dynamic range of imaging systems beyond that of the pixel itself. All of them have some kind of penalty however, (e.g. in resolution or frame rate) and they can introduce undesirable artifacts which become problematic in extreme cases. Reducing the sensitivity has the consequence that greater light power is required to bring the darker regions of the scene to acceptable signal levels. Lowering the F-number will compensate for a loss in sensitivity too, but at the cost of spatial distortion and reduced depth of focus.

With an image sensor located in the distal end of an endoscopic device, there are challenges present, which are not at issue when the imaging sensor is located remotely from the distal end of the endoscopic device. For example, when a user or operator rotates or changes the angle of the endoscopic device, which is common during a surgery, the image sensor will change orientation and the image horizon shown on screen will also change. What is needed are devices and systems that accommodate an image sensor being located in the distal end of the endoscopic device without changing the orientation and maintaining a constant image horizon for the user or operator. As will be seen, the disclosure provides devices and systems that can do this in an efficient and elegant manner.

### BRIEF DESCRIPTION OF THE DRAWINGS

Non-limiting and non-exhaustive implementations of the disclosure are described with reference to the following figures, wherein like reference numerals refer to like parts throughout the various views unless otherwise specified. Advantages of the disclosure will become better understood with regard to the following description and accompanying drawings where:

FIG. 1 is a side, cross-sectional view of an endoscopic system, illustrating a rigidly coupled image sensor located at a tip of the endoscope, and further illustrating a fixed inner lumen and a rotatable outer lumen according to one implementation;

FIG. 2 is a side, cross-sectional view of the endoscopic system of FIG. 1, illustrating the inner lumen and the outer lumen with their respective optical components in an exploded view;

FIG. 3 is an enlarged, detailed view of the tip of the endoscope illustrated in FIG. 1 according to one implementation;

FIG. 4 is an enlarged, detailed view of the tip of the endoscope according to one implementation;

FIG. 5 illustrates one implementation of the endoscopic device, illustrating the ability of the outer lumen, along with a distal lens and prism, of the endoscope to rotate while maintaining the position of the image sensor to create a wide angle field of vision;

FIG. 6 illustrates one implementation of the endoscopic device, where the outer lumen has been rotated one-hundred and eighty degrees with respect to the view in

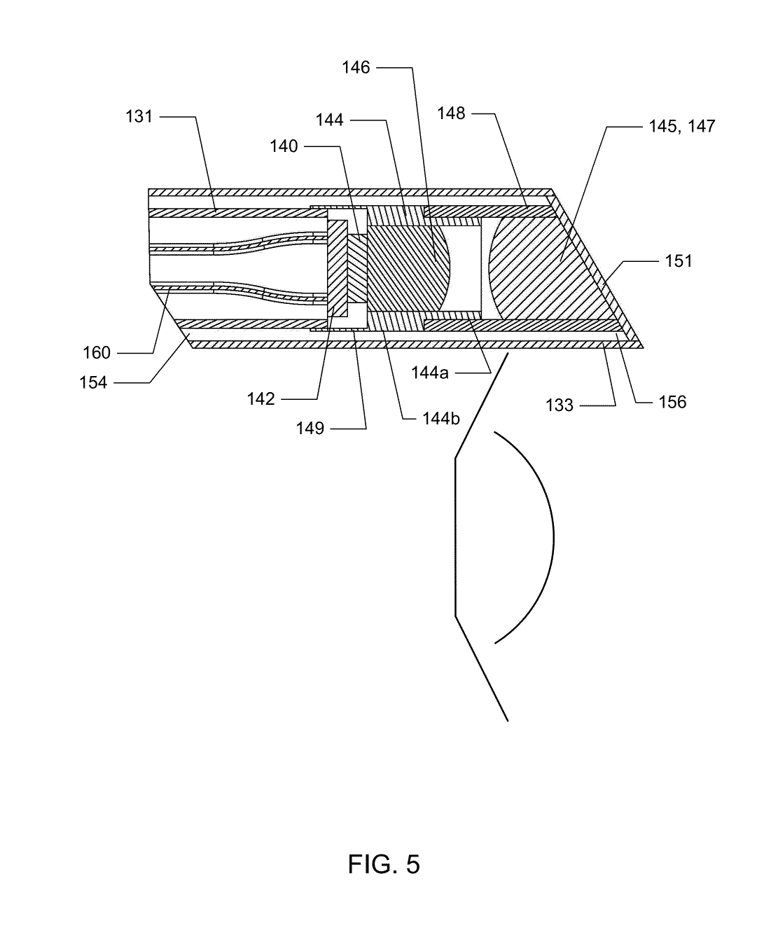

FIG. 5 and illustrating a limited field of view in comparison to FIG. 5 and according to one implementation;

FIGS. 7A and 7B illustrate a perspective view and a side view, respectively, of an implementation of a monolithic sensor having a plurality of pixel arrays for producing a three dimensional image in accordance with the teachings and principles of the disclosure;

FIGS. 8A and 8B illustrate a perspective view and a side view, respectively, of an implementation of an imaging sensor built on a plurality of substrates, wherein a plurality of pixel columns forming the pixel array are located on the first substrate and a plurality of circuit columns are located on a second substrate and showing an electrical connection and communication between one column of pixels to its associated or corresponding column of circuitry; and

FIGS. 9A and 9B illustrate a perspective view and a side view, respectively, of an implementation of an imaging sensor having a plurality of pixel arrays for producing a three dimensional image, wherein the plurality of pixel arrays and the image sensor are built on a plurality of substrates.

### DETAILED DESCRIPTION

The disclosure extends to endoscopic devices and systems for image rotation for a rigidly coupled image sensor. The disclosure allows for a distal prism to rotate, which changes the angle of view of the user or operator, while the sensor remains fixed at a constant location. This allows the device to be used in the same manner as expected by a user or operator experienced in using conventional rigid endoscopy systems. The user or operator may rotate an outer lumen, thereby changing the angle of view, while the sensor remains in a fixed position and the image viewable on screen remains at a constant horizon. The prism may rotate while the sensor does not rotate, such that the user does not lose orientation.

In the following description of the disclosure, reference is made to the accompanying drawings, which form a part hereof, and in which is shown by way of illustration specific implementations in which the disclosure may be practiced. It is understood that other implementations may be utilized and structural changes may be made without departing from the scope of the disclosure.

It must be noted that, as used in this specification and the appended claims, the singular forms “a,” “an,” and “the” include plural referents unless the context clearly dictates otherwise.

As used herein, the terms “comprising,” “including,” “containing,” “characterized by,” and grammatical equivalents thereof are inclusive or open-ended terms that do not exclude additional, unrecited elements or method steps.

Further, where appropriate, functions described herein can be performed in one or more of: hardware, software, firmware, digital components, or analog components. For example, one or more application specific integrated circuits (ASICs) can be programmed to carry out one or more of the systems and procedures described herein. Certain terms are used throughout the following description and Claims to refer to particular system components. As one skilled in the art will appreciate, components may be referred to by different names. This document does not intend to distinguish between components that differ in name, but not function.

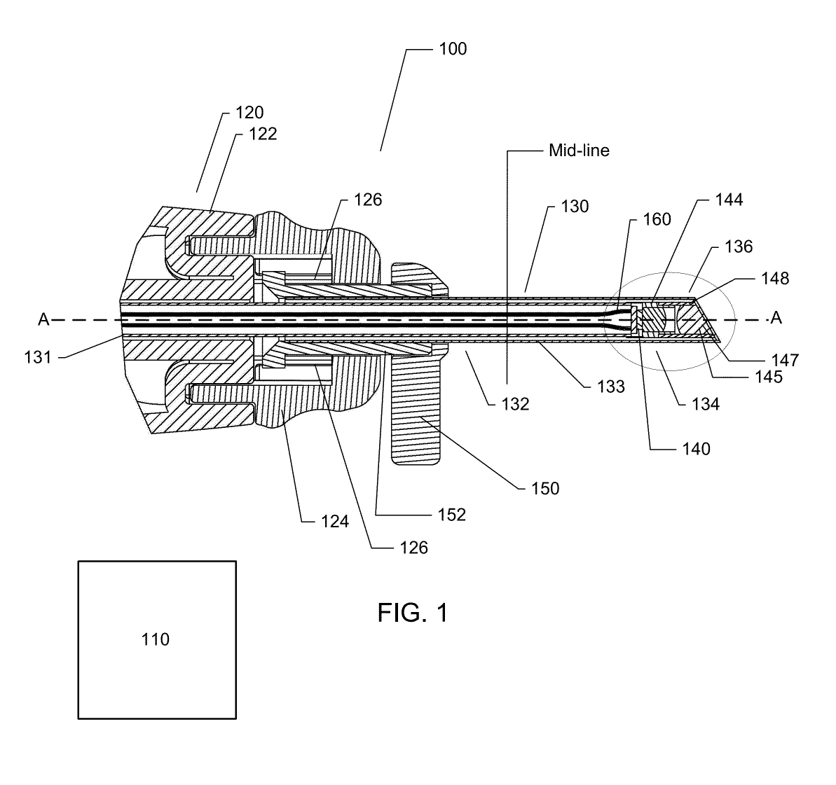

Referring now to the figures, it will be appreciated that FIG. 1 illustrates an example of an endoscopic system 100 according to the disclosure. The endoscopic system 100 may comprise a control unit 110, a handpiece 120, and an endoscopic device 130. It will be appreciated that the control unit 110 may be located remotely from an image sensor 140 (discussed more fully herein) and may be located in the handpiece 120 in an implementation. In one implementation the control unit 110 may be located remotely from the image sensor 140 and may be housed at a base unit without departing from the scope of the disclosure.

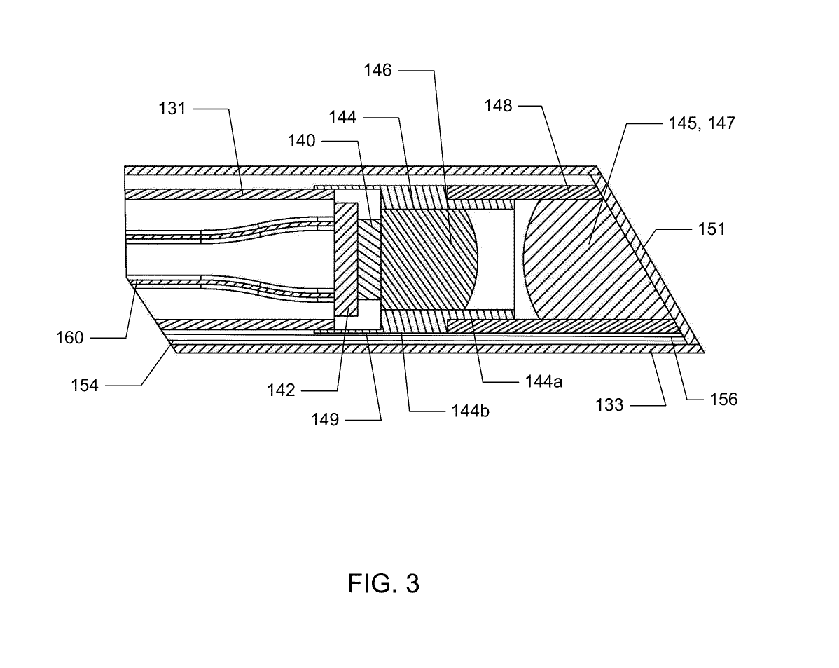

In one implementation, the handpiece 120 may comprise a body 122 that may be fixed relative and attached to an inner lumen 131 of the endoscopic device 130. The handpiece 120 may also comprise a spring loaded mechanism. The spring loaded mechanism may comprise a spring cap 124, which may be located adjacent the body 122. The spring cap 124 may be fixed and attached to the inner lumen 131 of the endoscope 130. At least one spring 126 may be present in the spring cap 124 and may be part of the spring loaded mechanism. This spring-loaded mechanism may function to maintain constant contact between a distal lens holder 148 and a proximal lens holder 144, discussed more fully below in relation to FIG. 3. The system 100 may also comprise a rotation post 150 that is attached to a spring sleeve 152. The spring sleeve 152 may be attached to the outer lumen 133, such that both the rotation post 150 and the spring sleeve 152 may be rotated relative to the inner lumen 131. As the rotation post 150 is moved, the spring 126 may operate to push against the spring cap 124 and spring sleeve 152 causing consistent contact between the distal lens holder 148 and the proximal lens holder 144. It will be appreciated that the spring 126 may operate to maintain axial pressure and ensure that there is a consistent distance between lens elements 146, thereby allowing rotation without axial movement and a loss of focus.

It will be appreciated that the outer lumen 133 may be in mechanical communication with the handpiece 120. In an implementation, the outer lumen 133 may be spring-loaded at a junction with the handpiece 120 to provide consistent contact between the distal lens holder 148 and the proximal lens holder 144, thus ensuring consistent axial distance with the proximal lens elements 146 and the distal lens elements 147 and retaining focus while the outer lumen 133 rotates.

In an implementation, the handpiece 120 may comprise a focus mechanism. The focus mechanism may permit focal adjustments in the system and may be attached to the inner lumen 131, such that the inner lumen 131 is movable axially as the focus mechanism may function to control the axial distance between the proximal lens 146 and the distal lens 147. The focus mechanism may move the inner lumen 131 in the axial direction only and may not allow rotation.

The endoscopic device 130 may comprise a proximal portion 132, which may be defined as the portion nearest the handpiece 120, and a distal portion 134, which may be defined as the portion farthest away from the handpiece 120. The distal portion 134 may comprise a tip 136. The endoscopic device 130 may house the image sensor 140 for providing visualization of an area. In one implementation, the image sensor 140 may be located within the distal portion 134 at or near the tip 136 of the endoscopic device 130. The endoscopic device may also comprise the inner lumen 131 and the outer lumen 133. In one implementation, the image sensor 140 and the inner lumen 131 may be fixed relative to the outer lumen 133. In the implementation, the outer lumen 133 may be rotatable about an axis A-A of the endoscope 130 and with respect to the image sensor 140 and the inner lumen 131. Thus, the disclosure extends to any endoscopic device and system for use with a rigidly coupled image sensor 140.

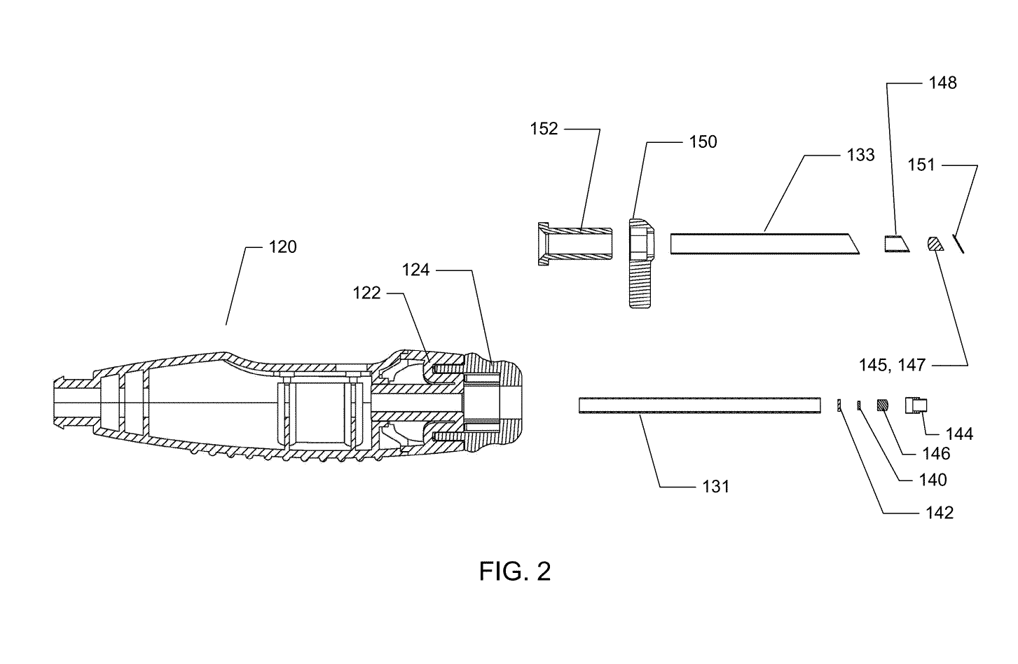

Referring now to FIG. 2, which is an exploded, side cross-sectional view of the endoscopic system of FIG. 1, the inner lumen 131 and the outer lumen 133 are illustrated with their respective optical components in an exploded view. As noted, the inner lumen 131 may be fixed relative to the handpiece 120. The image sensor 140 may be fixed to the inner lumen 131. In one implementation, the proximal lens holder 144 holds the proximal lens elements 146, the image sensor 140, and support hardware 142 and is fixed to the inner lumen 131. The proximal lens holder 144 may abut against the distal lens holder 148.

The distal lens holder 148 may be rotatable with respect to the inner lumen 131. It will be appreciated that the outer lumen 133 may be freely rotatable, such that any components that are attached thereto may also be free to rotate. The distal lens holder 148 may be attached to the outer lumen 133 and is freely rotatable. The distal lens holder 148 may abut against an outer window 151. The outer window 151 may also be attached to the outer lumen 133 and may be rotatable relative to the inner lumen 131 and the image sensor 140. The outer window 151 may be in mechanical communication with the outer lumen 133 and may be located on the terminal end of the tip 136 of the endoscope 130.

The distal lens holder 148 may house a prism 145 and a distal lens 147, both of which may be located at or near the tip 136 of the endoscope 130. It should be noted that the prism 145 as shown in the Figures and referenced herein may be comprised of multiple elements as necessary to properly change the direction of light through the system. It should also be noted the proximal lens 146 and distal lens 147 as shown in the Figures and referenced herein together comprise a complete lens system that projects a focused image on the image sensor 140. The lens system may be comprised of multiple elements and any number of these elements may be included in the distal lens 147 with the remainder included in the proximal lens 146. The prism 145 and the distal lens 147 may both be fixed to the outer lumen 133 and may be rotatable relative to the inner lumen 131 and the image sensor 140, such that as the angle of view is changed the orientation of an image remains constant within the viewing area of the user. It will be appreciated that the distal lens holder 148 may comprise a guide for aligning the prism 145 and the distal lens 147 within the tip 136 of the endoscope 130. The distal lens holder 148 may be fixed to the outer lumen 133 and may be rotatable relative to the inner lumen 131 and the image sensor 140. The distal lens 147 may be located near the tip 136 of the endoscope 130 and the proximal lens 146 may be located proximally with respect to the distal lens 147. The proximal lens 146 may be fixed to the inner lumen 131, such that it remains fixed relative to the outer lumen 133 as the outer lumen 133 is rotated.

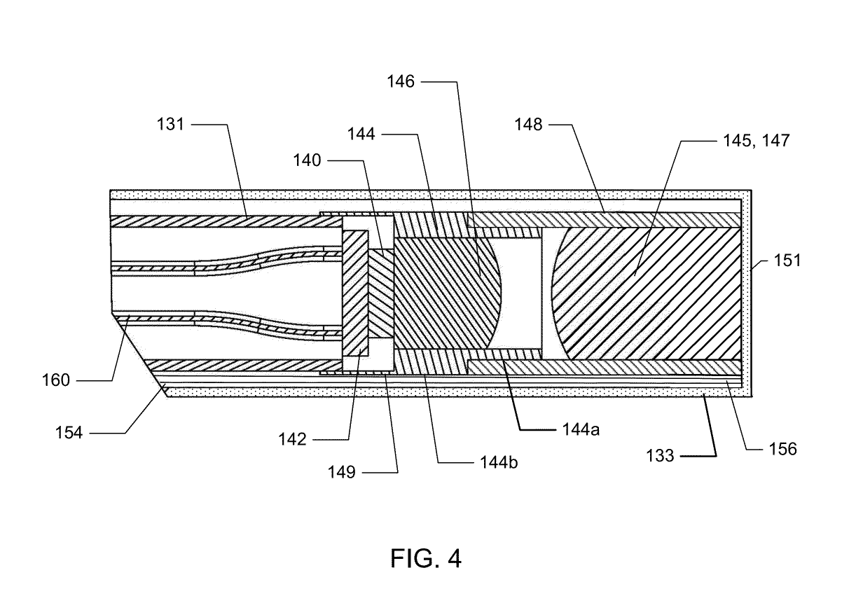

As illustrated in FIGS. 3 and 4, which are detailed views of alternative implementations of the distal portion 134 and tip 136 of the endoscope 130, a channel 154 may be formed between the inner lumen 131 and the outer lumen 133, wherein the channel 154 may house fiber optics 156 for providing a light source to the surgical scene. The fiber optics 156 may be fixed to the outer lumen 133 and may be rotatable relative to the inner lumen 131 and the image sensor 140. In an implementation, the endoscope 130 may further comprise a friction reducing layer formed between the outer lumen 133 and the inner lumen 131, such that friction is reduced between the inner lumen 131 and the outer lumen 133 to allow easy rotation. It will be appreciated that the friction reducing layer may be any material that provides lubrication to allow rotation of the outer lumen 133 with respect to the inner lumen 131.

The proximal lens holder 144 may comprise an inner guide wall 144a that is formed at one end of the proximal lens holder 144 and an outer guide wall 144b that is formed at the other end of the proximal lens holder 144. The proximal lens holder 144 acts as a housing and guide for aligning the proximal lens 146 with respect to the distal lens 147, wherein the proximal lens holder 144 is fixed to the inner lumen 131 and remains fixed relative to the outer lumen 133 as the outer lumen 133 is rotated. In an implementation, the inner guide wall 144a may engage the guide of the distal lens holder 148, such that the distal lens holder 148 is rotatable with respect to the proximal lens holder 144.

In one implementation, as illustrated in FIG. 3, the outer window 151 may be formed at an angle. The angle may be any angle that may be useful in endoscopy and may fall within a range of about zero degrees to about ninety degrees, and may be about thirty degrees. However, it will be appreciated that in one implementation the outer window 151 may comprise a zero angle as illustrated in FIG. 4 without departing from the scope of the disclosure. It will be appreciated that all outer window angles that fall within the above-noted range of about zero degrees to about ninety degrees fall within the scope of the disclosure as if each angle were independently identified herein, such that the scope of the disclosure includes all angles within the identified range. For example, angles of about five degrees, about ten degrees, about fifteen degrees, about twenty degrees, about twenty-five degrees, about thirty degrees, about thirty-five degrees, about forty degrees, about forty-five degrees, about fifty degrees, about fifty-five degrees, about sixty degrees, about sixty-five degrees, about seventy degrees, about seventy-five degrees, about eighty degrees, and about eighty-five degrees and all angles in between about zero and about ninety degrees fall within the scope of the disclosure.

As illustrated best in FIGS. 3 and 4, the endoscopic device 130 may further comprise an electrical communication harness 160. The harness 160 may be fixed to and located within the inner lumen 131. The electrical communication harness 160 may be electrically connected to or in communication with the image sensor 140, thereby providing power to the image sensor 140. Because of its association and connection to the inner lumen 131, the electrical communication harness 160 may be fixed relative to the outer lumen.

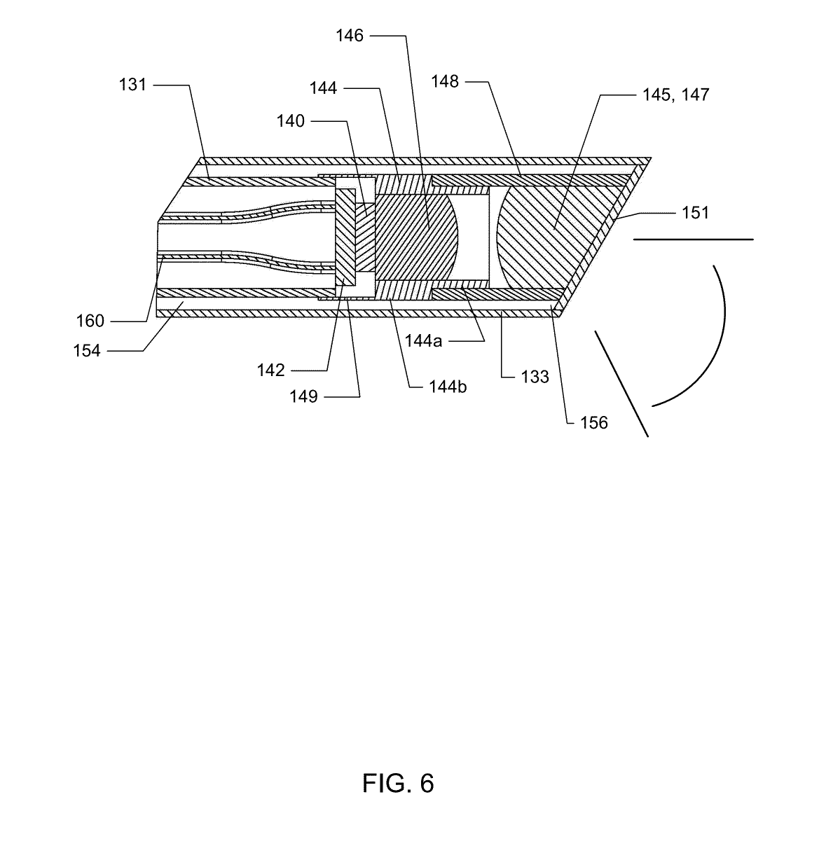

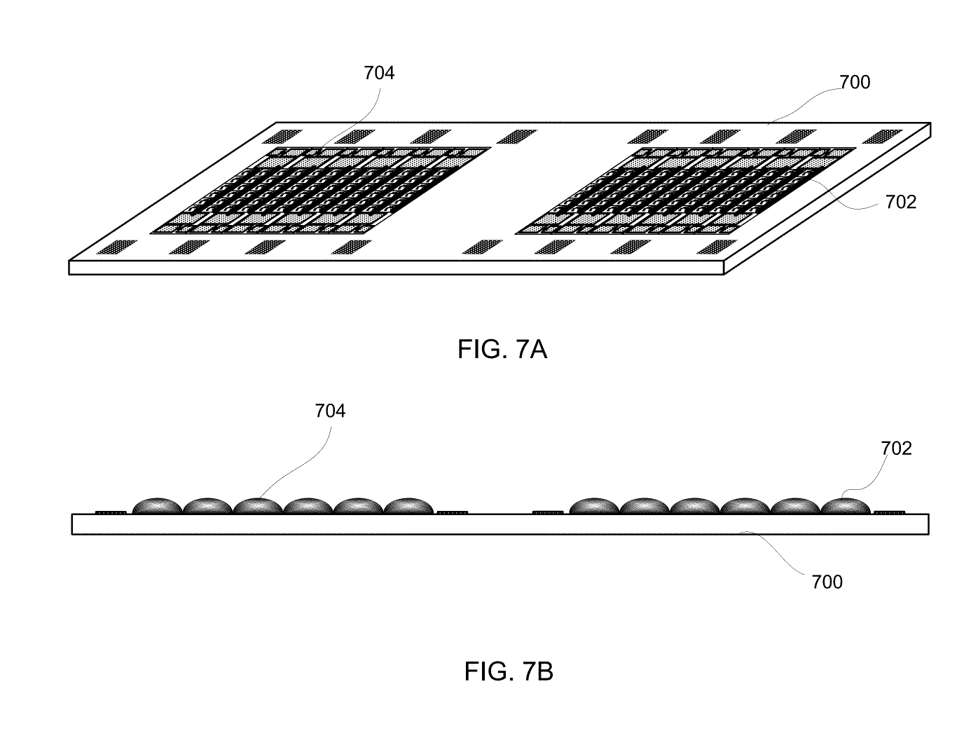

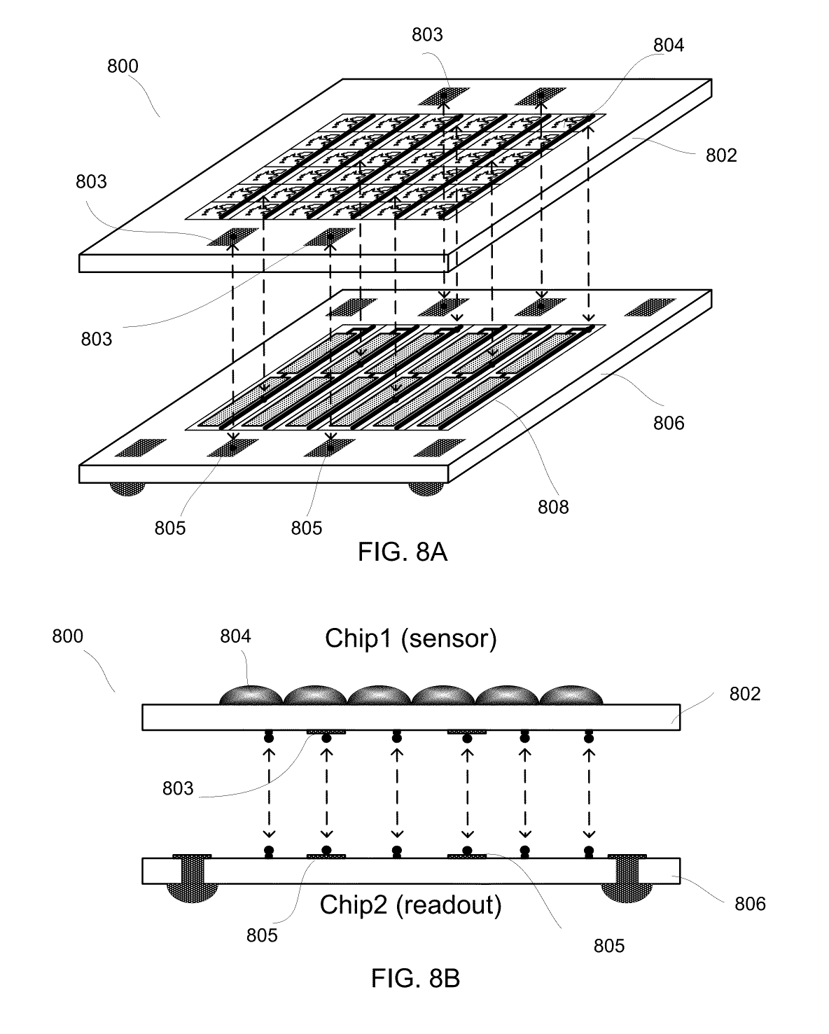

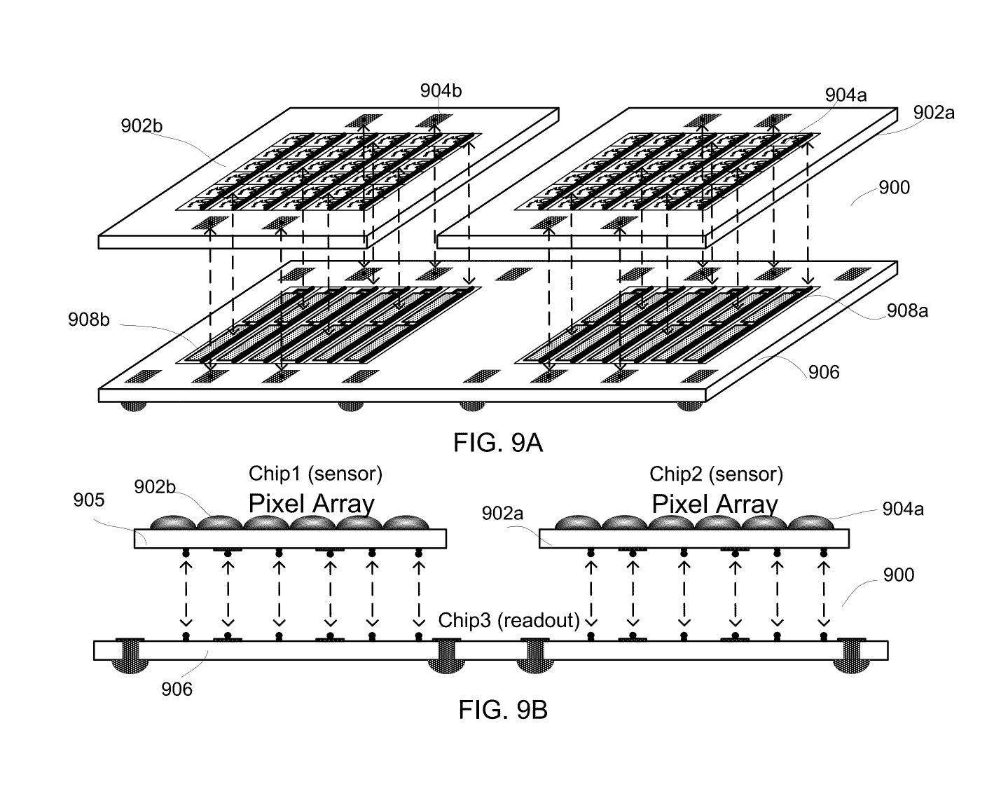

Referring now to FIGS. 5 and 6, there is illustrated the ability of the outer lumen 133 and the distal lens 147 and prism 145 of the endoscope 130 to rotate while maintaining the positioning of the image sensor 140. The rotation ability provides the advantage of creating a wide angle field of vision without creating distortion as seen in a fisheye lens. It will be appreciated that because of the rotation of the distal prism 145, the angle of view of the user or operator is changed accordingly, while the sensor 140 remains fixed at a constant location. This allows the endoscopic device 130 to be used in the same manner as expected by a user or operator using a traditional endoscope. The user or operator may rotate the outer lumen 133, thereby changing the angle of view, while the sensor 140 remains in a fixed position and the image viewable on screen remains at a constant horizon. The prism 145 may rotate while the sensor 140 does not rotate, such that the user does not lose orientation.

Referring generally to the image sensor technology illustrated in FIGS. 7A-9B, and referring to sensor technology generally, it will be appreciated that CMOS image sensors have largely displaced conventional CCD imagers in modern camera applications such as endoscopy, owing to their greater ease of integration and operation, superior or comparable image quality, greater versatility, and lower cost.

Typically CMOS image sensors include the circuitry necessary to convert the image information into digital data and have various levels of digital processing incorporated thereafter. This can range from basic algorithms for the purpose of correcting non-idealities, which may, for example, arise from variations in amplifier behavior to full image signal processing (ISP) chains, providing video data in the standard sRGB color space (cameras-on-chip).

The desired degree of sensor complexity for a given camera system is driven by several factors, one of which is the available physical space for the image sensor. The most extreme functionally minimal CMOS sensor would have only the basic pixel array plus a degree of buffering to drive the analog data off chip. All of the timing signals required to operate and read out the pixels would be provided externally. The need to supply the control signals externally adds many pads, which consume significant real estate, however. Therefore it doesn't necessarily follow that minimal functionality equates to minimal area.

If the second stage is an appreciable distance from the sensor, it becomes much more desirable to transmit the data in the digital domain, since it is rendered immune to interference noise and signal degradation. There is a strong desire to minimize the number of conductors since that reduces the number of pads on the sensor (which consume space), plus the complexity and cost of camera manufacture. Although the addition of analog to digital conversion to the sensor is necessitated, the additional area is offset to a degree, owing to a significant reduction in the required analog buffering power. In terms of area consumption, given the typical feature size available in computer information systems technologies, it is preferable to have all of the internal logic signals be generated on chip via a set of control registers and a simple command interface.

The disclosure contemplates and covers aspects of a combined sensor and system design that allows for high definition imaging with reduced pixel counts in a highly controlled illumination environment. This is accomplished by virtue of frame by frame pulsed color switching at the light source in conjunction with high frames capture rates and a specially designed monochromatic sensor. Since the pixels are color agnostic, the effective spatial resolution is appreciably higher than for their color (usually Bayer-pattern filtered) counterparts in conventional single-sensor cameras. They also have higher quantum efficiency since far fewer incident photons are wasted. Moreover, Bayer based spatial color modulation requires that the modulation transfer function (MTF) of the accompanying optics be lowered compared with the monochrome case, in order to blur out the color artifacts associated with the Bayer pattern. This has a detrimental impact on the actual spatial resolution that can be realized with color sensors.

The disclosure is also concerned with a system solution for endoscopy applications in which the image sensor is resident at the distal end of the endoscope. In striving for a minimal area sensor based system, there are other design aspects that can be developed too, beyond the obvious reduction in pixel count. In particular, the area of the digital portion of the chip should be minimized, as should the number of connections to the chip (pads). This involves the design of a full-custom CMOS image sensor with several novel features.

It will be appreciated that the disclosure may be used with any image sensor, whether a CMOS image sensor or CCD image sensor, without departing from the scope of the disclosure. Further, the image sensor may be located in any location within the overall system, including, but not limited to, the tip of the endoscope, the hand piece of the imaging device or camera, the control unit, or any other location within the system without departing from the scope of the disclosure.

Implementations of an image sensor that may be utilized by the disclosure include, but are not limited to, the following, which are merely examples of various types of sensors that may be utilized by the disclosure.

Referring now to FIGS. 7A and 7B, the figures illustrate a perspective view and a side view, respectively, of an implementation of a monolithic sensor 700 having a plurality of pixel arrays for producing a three dimensional image in accordance with the teachings and principles of the disclosure. Such an implementation may be desirable for three dimensional image capture, wherein the two pixel arrays 702 and 704 may be offset during use. In another implementation, a first pixel array 702 and a second pixel array 704 may be dedicated to receiving a predetermined range of wave lengths of electromagnetic radiation, wherein the first pixel array 702 is dedicated to a different range of wave length electromagnetic radiation than the second pixel array 704.

FIGS. 8A and 8B illustrate a perspective view and a side view, respectively, of an implementation of an imaging sensor 800 built on a plurality of substrates. As illustrated, a plurality of pixel columns 804 forming the pixel array are located on the first substrate 802 and a plurality of circuit columns 808 are located on a second substrate 806. Also illustrated in the figure are the electrical connection and communication between one column of pixels to its associated or corresponding column of circuitry. In one implementation, an image sensor, which might otherwise be manufactured with its pixel array and supporting circuitry on a single, monolithic substrate/chip, may have the pixel array separated from all or a majority of the supporting circuitry. The disclosure may use at least two substrates/chips, which will be stacked together using three-dimensional stacking technology. The first 802 of the two substrates/chips may be processed using an image CMOS process. The first substrate/chip 802 may be comprised either of a pixel array exclusively or a pixel array surrounded by limited circuitry. The second or subsequent substrate/chip 806 may be processed using any process, and does not have to be from an image CMOS process. The second substrate/chip 806 may be, but is not limited to, a highly dense digital process in order to integrate a variety and number of functions in a very limited space or area on the substrate/chip, or a mixed-mode or analog process in order to integrate for example precise analog functions, or a RF process in order to implement wireless capability, or MEMS (Micro-Electro-Mechanical Systems) in order to integrate MEMS devices. The image CMOS substrate/chip 802 may be stacked with the second or subsequent substrate/chip 806 using any three-dimensional technique. The second substrate/chip 806 may support most, or a majority, of the circuitry that would have otherwise been implemented in the first image CMOS chip 802 (if implemented on a monolithic substrate/chip) as peripheral circuits and therefore have increased the overall system area while keeping the pixel array size constant and optimized to the fullest extent possible. The electrical connection between the two substrates/chips may be done through interconnects 803 and 805, which may be wirebonds, bump and/or TSV (Through Silicon Via).

FIGS. 9A and 9B illustrate a perspective view and a side view, respectively, of an implementation of an imaging sensor 900 having a plurality of pixel arrays for producing a three dimensional image. The three dimensional image sensor may be built on a plurality of substrates and may comprise the plurality of pixel arrays and other associated circuitry, wherein a plurality of pixel columns 904a forming the first pixel array and a plurality of pixel columns 904b forming a second pixel array are located on respective substrates 902a and 902b, respectively, and a plurality of circuit columns 908a and 908b are located on a separate substrate 906. Also illustrated are the electrical connections and communications between columns of pixels to associated or corresponding column of circuitry.

It will be appreciated that the teachings and principles of the disclosure may be used in a reusable device platform, a limited use device platform, a re-posable use device platform, or a single-use/disposable device platform without departing from the scope of the disclosure. It will be appreciated that in a re-usable device platform an end-user is responsible for cleaning and sterilization of the device. In a limited use device platform the device can be used for some specified amount of times before becoming inoperable. Typical new device is delivered sterile with additional uses requiring the end-user to clean and sterilize before additional uses. In a re-posable use device platform a third-party may reprocess the device (e.g., cleans, packages and sterilizes) a single-use device for additional uses at a lower cost than a new unit. In a single-use/disposable device platform a device is provided sterile to the operating room and used only once before being disposed of.

Additionally, the teachings and principles of the disclosure may include any and all wavelengths of electromagnetic energy, including the visible and non-visible spectrums, such as infrared (IR), ultraviolet (UV), and X-ray.

The foregoing description has been presented for the purposes of illustration and description. It is not intended to be exhaustive or to limit the disclosure to the precise form disclosed. Many modifications and variations are possible in light of the above teaching. Further, it should be noted that any or all of the aforementioned alternate implementations may be used in any combination desired to form additional hybrid implementations of the disclosure.

Further, although specific implementations of the disclosure have been described and illustrated, the disclosure is not to be limited to the specific forms or arrangements of parts so described and illustrated. The scope of the disclosure is to be defined by the claims appended hereto, any future claims submitted here and in different applications, and their equivalents.

What is claimed is:

1. An endoscopic device comprising:
a proximal portion and a distal portion, wherein the distal portion comprises a tip;
an image sensor for providing visualization of an area, wherein the image sensor is located within the distal portion near the tip of the endoscopic device;
an inner lumen; and
an outer lumen;
wherein the image sensor and the inner lumen are fixed relative to the outer lumen, and wherein the outer lumen is rotatable about an axis of the endoscope and with respect to the image sensor and the inner lumen.

2. The endoscopic device of claim 1, wherein the device further comprises a channel formed between the inner lumen and the outer lumen, wherein the channel houses fiber optics for providing a light source, and wherein the fiber optics are fixed to the outer lumen and are rotatable relative to the inner lumen and the image sensor.

3. The endoscopic device of claim 1, wherein the device further comprises a channel formed between the inner lumen and the outer lumen, wherein the channel houses fiber optics for providing a light source, and wherein the fiber optics are fixed to the inner lumen, such that the outer lumen is rotatable with respect to the inner lumen and the fiber optics.

4. The endoscopic device of claim 1, wherein the device further comprises a prism and a distal lens that are located near the tip of the endoscope, wherein the prism and the distal lens are fixed to the outer lumen and are rotatable relative to the inner lumen and the image sensor, such that as the angle of view is changed the orientation of the image remains constant.

5. The endoscopic device of claim 4, wherein the device further comprises a distal lens holder comprising a guide for aligning the prism and the distal lens within the tip of the endoscope, wherein the distal lens holder is fixed to the outer lumen and is rotatable relative to the inner lumen and the image sensor.

6. The endoscopic device of claim 1, wherein the device further comprises a proximal lens and a distal lens, wherein the distal lens is located near the tip of the endoscope and the proximal lens is located proximally with respect to the distal lens, and wherein the proximal lens is fixed to the inner lumen, such that it remains fixed relative to the outer lumen as said outer lumen is rotated.

7. The endoscopic device of claim 5, wherein the device further comprises a proximal lens holder comprising an inner guide wall that is formed at one end of the proximal lens holder and an outer guide wall that is formed at the other end of the proximal lens holder, wherein the proximal lens holder acts as a housing and guide for aligning the proximal lens with respect to the distal lens, wherein the proximal lens holder is fixed to the inner lumen and remains fixed relative to the outer lumen as said outer lumen is rotated;
wherein the inner guide wall of the proximal lens holder engages the guide of the distal lens holder, such that the distal lens holder is rotatable with respect to the proximal lens holder.

8. The endoscopic device of claim 1, wherein the device further comprises an outer window that is in mechanical communication with the outer lumen and is located on the tip of the endoscope, wherein the outer window rotates relative to the inner lumen and the image sensor.

9. The endoscopic device of claim 1, wherein the device further comprises a friction reducing layer formed between the outer lumen and the inner lumen to thereby reduce the friction therebetween, such that friction is reduced thereby allowing the outer lumen to rotate with respect to the inner lumen.

10. The endoscopic device of claim 1, wherein the device further comprises an electrical communication harness that is fixed to the inner lumen and located within the inner lumen, wherein the electrical communication harness is in electrically connected to the image sensor, thereby providing power to said image sensor, wherein the electrical communication harness is fixed relative to the outer lumen.

11. The endoscopic device of claim 7, wherein the device further comprises a handpiece, wherein the outer lumen is in mechanical communication with the handpiece, and wherein the outer lumen is spring-loaded at a junction with the handpiece to provide consistent contact between the distal lens holder and the proximal lens holder, thus ensuring consistent axial distance within the proximal lens and the distal lens and retaining focus while the outer lumen is rotating.

12. The endoscopic device of claim 6, wherein the device further comprises a handpiece with a focus mechanism, wherein the inner lumen is movable axially via the focus mechanism within the handpiece to allow for focal adjustments, wherein the focus mechanism controls the axial distance between the proximal lens and the distal lens, and wherein the focus mechanism moves the inner lumen in the axial direction only and does not allow rotation.

13. The endoscopic device of claim 4, wherein the rotation of the outer lumen, the distal lens and the prism of the endoscope creates the effect of a wide angle field of vision without distortion.

14. An endoscopic system comprising:
a handpiece;
a control unit;
an endoscope device comprising:
a proximal portion and a distal portion, wherein the distal portion comprises a tip;
an image sensor for providing visualization of an area, wherein the image sensor is located within the distal portion near the tip of the endoscopic device;
an inner lumen; and
an outer lumen;
wherein the image sensor and the inner lumen are fixed relative to the outer lumen, and wherein the outer lumen is rotatable about an axis of the endoscope and with respect to the image sensor and the inner lumen.

15. The endoscopic system of claim 14, wherein the endoscopic device further comprises a channel formed between the inner lumen and the outer lumen, wherein the channel houses fiber optics for providing a light source, and wherein the fiber optics are fixed to the outer lumen and are rotatable relative to the inner lumen and the image sensor.

16. The endoscopic device of claim 14, wherein the device further comprises a channel formed between the inner lumen and the outer lumen, wherein the channel houses fiber optics for providing a light source, and wherein the fiber optics are fixed to the inner lumen, such that the outer lumen is rotatable with respect to the inner lumen and the fiber optics.

17. The endoscopic system of claim 14, wherein the endoscopic device further comprises a prism and a distal lens that are located near the tip of the endoscope, wherein the prism and the distal lens are fixed to the outer lumen and are rotatable relative to the inner lumen and the image sensor, such that as the angle of view is changed the orientation of the image remains constant.

18. The endoscopic system of claim 17, wherein the endoscopic device further comprises a distal lens holder comprising a guide for aligning the prism and the distal lens within the tip of the endoscope, wherein the distal lens holder is fixed to the outer lumen and is rotatable relative to the inner lumen and the image sensor.

19. The endoscopic system of claim 14, wherein the endoscopic device further comprises a proximal lens and a distal lens, wherein the distal lens is located near the tip of the endoscope and the proximal lens is located proximally with respect to the distal lens, and wherein the proximal lens is fixed to the inner lumen, such that it remains fixed relative to the outer lumen as said outer lumen is rotated.

20. The endoscopic system of claim 18, wherein the endoscopic device further comprises a proximal lens holder comprising an inner guide wall that is formed at one end of the proximal lens holder and an outer guide wall that is formed at the other end of the proximal lens holder, wherein the proximal lens holder acts as a housing and guide for aligning the proximal lens with respect to the distal lens, wherein the proximal lens holder is fixed to the inner lumen and remains fixed relative to the outer lumen as said outer lumen is rotated;
wherein the inner guide wall of the proximal lens holder engages the guide of the distal lens holder, such that the distal lens holder is rotatable with respect to the proximal lens holder.

21. The endoscopic system of claim 14, wherein the endoscopic device further comprises an outer window that is in mechanical communication with the outer lumen and is located on the tip of the endoscope, wherein the outer window rotates relative to the inner lumen and the image sensor.

22. The endoscopic system of claim 14, wherein the endoscopic device further comprises a friction reducing layer formed between the outer lumen and the inner lumen to thereby reduce the friction therebetween, such that friction is reduced thereby allowing the outer lumen to rotate with respect to the inner lumen.

23. The endoscopic system of claim 14, wherein the endoscopic device further comprises an electrical communication harness that is fixed to the inner lumen and located within the inner lumen, wherein the electrical communication harness is in electrically connected to the image sensor, thereby providing power to said image sensor, wherein the electrical communication harness is fixed relative to the outer lumen.

24. The endoscopic system of claim 20, wherein the endoscopic device further comprises a handpiece, wherein the outer lumen is in mechanical communication with the handpiece, and wherein the outer lumen is spring-loaded at a junction with the handpiece to provide consistent contact between the distal lens holder and the proximal lens holder, thus ensuring consistent axial distance within the proximal lens and the distal lens and retaining focus while the outer lumen is rotating.

25. The endoscopic system of claim 19, wherein the endoscopic device further comprises a handpiece and a focus mechanism, wherein the inner lumen is movable axially via the focus mechanism in the handpiece to allow for focal adjustments, wherein the focus mechanism controls the axial distance between the proximal lens and the distal lens, and wherein the focus mechanism moves the inner lumen in the axial direction only and does not allow rotation.

26. The endoscopic system of claim 17, wherein the rotation of the outer lumen, the distal lens and the prism of the endoscope creates the effect of a wide angle field of vision without distortion.
